(19) United States
(12) Patent Application Publication
(10) Pub. No.: US 2009/0030091 A1
(43) Pub. Date: Jan. 29, 2009

(54) Brown Film Composition and Method for Preparing the Same

(75) Inventors: Sumihiro Shiraishi, Yamatokoriyama-shi (JP); Yusuke Suzuki, Yamatokoriyama-shi (JP)

Correspondence Address: FITCH, EVEN, TABIN & FLANNERY, P. O. BOX 18415, WASHINGTON, DC, 20036, US

(73) Assignee: QUALICAPS CO., LTD., Yamatokoriyama-shi (JP)

(21) Appl. No.: 11/972,196

(22) Filed: Jan. 10, 2008

Related U.S. Application Data
(60) Provisional application No. 60/880,027, filed on Jan. 12, 2007.

(51) Int. Cl. A61K 47/38; A23L 1/27

(52) U.S. Cl. 514/781; 426/250

(57) ABSTRACT

The present invention provides a film composition containing caramel (Caramel I, II, III or IV) as a brown colorant, particularly a film composition containing a water-soluble cellulose derivative as a base component and suitably used for a capsule base, the film composition ensuring high degree of transparency because of the suppression of caramel aggregation. The present invention also provides a method for preparing the brown film composition. The present invention is enforced by adjusting pH of an aqueous solution in forming a film composition, which is obtained by solidification of an aqueous solution containing a water-soluble cellulose derivative and caramel, to a predetermined range using a pH adjuster.

### TECHNICAL FIELD

The present invention relates to a brown film composition containing caramel (Caramel I, II, III or IV) as a brown colorant. The present invention particularly relates to a film composition suitably used for a capsule base, the film composition being prepared using a water-soluble cellulose derivative and ensuring a high degree of transparency because of the suppression of caramel aggregation. The present invention also provides a method for preparing the brown film composition. The present invention further relates to a method for suppressing caramel aggregation in a brown film composition containing a water-soluble cellulose derivative as a film formation material, and caramel (Caramel I, II, III or IV) as a brown colorant.

### BACKGROUND ART

Gelatin capsules are most commonly used as hard capsules for medicine, quasi drugs, food etc. However, the strength of the gelatin capsules significantly decreases when the moisture content of the film shell decreases to 11% or less. Therefore, when the gelatin capsule is filled with a hygroscopic substance, the moisture in the capsule is absorbed by the substance, making the capsule fragile and prone to breaking. This defect causes difficulty in incorporating hygroscopic substances in a gelatin capsule. In particular, even though low-molecular-weight polyethylene glycols (hereafter occasionally referred to as “low-molecular-weight PEG”) whose average molecular weight ranges from 200 to 600, glycerin fatty esters and medium-chain triglycerides are widely used as diluents because of their excellent solubility and absorbency, drug formulation with these substances using a gelatin capsule is considered difficult because of their hygroscopicity. This problem also restricts the reduction of the moisture content of the gelatin capsule, and therefore a general relatively high. For this reason, gelatin capsules are not suitable to contain substances reactive with moisture or substances susceptible to moisture (see Patent Documents 1, 2 etc.).

To address this defect of conventional gelatin capsules, some nongelatin hard capsules, including a capsule containing a water-soluble cellulose derivative as a base material, and a capsule made of a water-soluble cellulose derivative mixed with polyvinyl alcohol or a gelatinizer (see Patent Documents 1 to 5, etc.), have been suggested. In particular, a capsule (HPMC capsule) comprising a hydroxy propyl methyl cellulose (HPMC) as a water-soluble cellulose derivative has desirable strength despite its low-moisture content, and can therefore contain a hygroscopic substance or a substance highly reactive with water.

Patent Document 1: U.S. application Ser. No. 07/957,892
Patent Document 2: U.S. application Ser. No. 08/114,351
Patent Document 3: U.S. application Ser. No. 04/833,715
Patent Document 4: U.S. application Ser. No. 06/946,119
Patent Document 5: U.S. application Ser. No. 07/266,060
Patent Document 6: U.S. application Ser. No. 10/157,428
Patent Document 7: U.S. application Ser. No. 09/240,504
Patent Document 8: U.S. application Ser. No. 10/941,182
Patent Document 9: U.S. application Ser. No. 10/865,409

### DISCLOSURE OF THE INVENTION

As described above, the water-soluble cellulose derivative is useful for a base of a hard capsule. However, an experiment conducted by the inventor of the present invention found that, when the water-soluble cellulose derivative is mixed with caramel that serves as a brown colorant, the reaction between the caramel and the water-soluble cellulose derivative causes caramel aggregation. The aggregation may appear as spots on the capsule base, or may decrease its transparency. Caramel is a conventionally known colorant for a capsule base (see Patent Documents 6 to 9 above, for example).

The present invention is made in view of the foregoing problem, and an object of the present invention is to provide a film composition that is prepared using a water-soluble cellulose derivative as a film formation material, and caramel (Caramel I, II, III or IV) as a brown colorant, particularly to a film composition suitable for a capsule base, the film composition ensuring a high degree of transparency because of the suppressed caramel aggregation. The present invention also provides a method for preparing the brown film composition, particularly a method that can suppress caramel aggregation. The present invention further provides a method for suppressing aggregation of caramel in a brown film composition prepared using a water-soluble cellulose derivative as a film formation material, and caramel (Caramel I, II, III or IV) as a brown colorant.

The inventors of the present invention conducted intensive study to achieve the foregoing object, and found that the problems of caramel aggregation etc. can be solved by using a pH adjuster, in addition to the water-soluble cellulose derivative and the caramel as a brown colorant, to prepare the film composition, so that the pH of the film composition falls within a desired range. This method allows for the preparation of a brown film composition with a high degree of transparency, and that is particularly suitable for a capsule base.

The present invention was completed based on such findings. The present invention includes the following matters.

### (1) Brown Film Composition

(1-1) A brown film composition in the shape of a film or a sheet, which comprises a water-soluble cellulose derivative, caramel, and a pH adjuster.

(1-2) A brown film composition according to (1-1), which is obtained by solidification of an aqueous solution containing a water-soluble cellulose derivative and caramel, wherein the aqueous solution forming a film contains a pH adjuster.

(1-3) A brown film composition according to (1-1) or (1-2), wherein the caramel is at least one selected from the group consisting of Caramel I, Caramel II, and Caramel IV, and wherein the pH adjuster is an acid agent.

(1-4) A brown film composition according to (1-2), wherein the caramel is at least one selected from the group consisting of Caramel I, Caramel II, and Caramel IV, and wherein the aqueous solution forming a film contains the pH adjuster so that the aqueous solution has a lower pH than an aqueous solution identical in composition except for the incorporation of the pH adjuster.

(1-5) A brown film composition according to (1-4), wherein the pH of the aqueous solution forming a film is not more than 7, preferably not more than 6.9.

(1-6) A brown film composition according to (1-1) or (1-2), wherein the caramel is Caramel III, and wherein the pH adjuster is an alkaline agent.

(1-7) A brown film composition according to (1-2), wherein the caramel is Caramel III, and wherein the aqueous solution forming a film contains the pH adjuster so that the aqueous solution has a higher pH than an aqueous solution identical in composition except for the incorporation of the pH adjuster.

(1-8) A brown film composition according to (1-7), wherein the pH of the aqueous solution forming a film is not less than 7.6, preferably not less than 7.7.

(1-9) A brown film composition according to (1-2), wherein the aqueous solution forming a film contains a gelatinizer and/or an auxiliary gelatinizer as required.

### (2) Container and Capsule Formulation

(2-1) A container for food, medicine, cosmetics, agrichemicals, or feed, which comprises a brown film composition of any one of (1-1) to (1-9) at least partially.

(2-2) A container according to (2-1), which is a form of a capsule.

(2-3) A capsule comprising a cap and a body, wherein at least one of the cap and the body comprises the brown film composition of any one of (1-1) to (1-9).

(2-4) A capsule formulation in which the capsule of (2-2) or (2-3) contains a filling.

(2-5) A capsule formulation according to (2-4), wherein the filling is food, medicine, cosmetics, agrichemicals, or feed.

(3) Preparation Method for Brown Film Composition

(3-1) A method for preparing a brown film composition, said method comprising solidifying an aqueous solution containing a water-soluble cellulose derivative, caramel, and a pH adjuster.

(3-2) A method according to (3-1), wherein the caramel is at least one selected from the group consisting of Caramel I, Caramel II, and Caramel IV, and wherein the pH adjuster is an acid agent.

(3-3) A method according to (3-2), wherein the caramel is at least one selected from the group consisting of Caramel I, Caramel II, and Caramel IV, and wherein the aqueous solution containing a water-soluble cellulose derivative, caramel, and a pH adjuster is adjusted by using the pH adjuster in such a quantity that the aqueous solution has a lower pH than an aqueous solution identical in composition except for the incorporation of the pH adjuster.

(3-4) A method according to (3-3), comprising adjusting the pH of the aqueous solution containing a water-soluble cellulose derivative, caramel, and a pH adjuster, to be not more than 7, preferably not more than 6.9.

(3-5) A method according to (3-1), wherein the caramel is Caramel III, and wherein the pH adjuster is an alkaline agent.

(3-6) A method according to (3-5), wherein the caramel is at least one selected from the group consisting of Caramel I, Caramel II, and Caramel IV, and wherein the aqueous solution containing a water-soluble cellulose derivative, caramel, and a pH adjuster is adjusted by using the pH adjuster in such a quantity that the aqueous solution has a higher pH than an aqueous solution identical in composition except for the incorporation of the pH adjuster.

(3-7) A method according to (3-6), comprising adjusting the pH of the aqueous solution containing a water-soluble cellulose derivative, caramel, and a pH adjuster, to be not less than 7.6, preferably not less than 7.7.

(3-8) A method according to (3-1), wherein the aqueous solution containing a water-soluble cellulose derivative, caramel, and a pH adjuster contains a gelatinizer and/or an auxiliary gelatinizer as required.

(3-9) A method according to any one of (3-1) to (3-8), which comprises suppressing caramel aggregation in the film composition.

### (4) Method for Suppressing the Generation of Aggregate of Caramel in a Film Composition

(4-1) A method for suppressing generation of aggregate of caramel in a film composition, which is obtained by solidification of an aqueous solution containing a water-soluble cellulose derivative and caramel, wherein the aqueous solution forming a film contains a pH adjuster.

(4-2) A method according to (4-1), wherein the caramel is at least one selected from the group consisting of Caramel I, Caramel II, and Caramel IV, and wherein the pH adjuster is an acid agent.

(4-3) A method according to (4-1), wherein the caramel is at least one selected from the group consisting of Caramel I, Caramel II, and Caramel IV, and wherein the aqueous solution forming a film contains the pH adjuster so that the aqueous solution has a lower pH than an aqueous solution identical in composition except for the incorporation of the pH adjuster.

(4-4) A method according to (4-3), wherein the pH of the aqueous solution forming a film is not more than 7, preferably not more than 6.9.

(4-5) A method according to (4-1), wherein the caramel is Caramel III, and wherein the pH adjuster is an alkaline agent.

(4-6) A method according to (4-1), wherein the caramel is Caramel III, and wherein the aqueous solution forming a film contains the pH adjuster so that the aqueous solution has a higher pH than an aqueous solution identical in composition except for the incorporation of the pH adjuster.

(4-7) A method according to (4-6), wherein the pH of the aqueous solution forming a film is not less than 7.6, preferably not less than 7.7.

(4-8) A method according to (4-1), wherein the aqueous solution contains a gelatinizer and/or an auxiliary gelatinizer as required.

### EFFECT OF THE INVENTION

The method of the present invention achieves significant suppression of the generation of aggregate of caramel used as a colorant in the production of a film composition using a water-soluble cellulose derivative. With this effect, the method prevents spots or turbidity on the capsule base, and thereby produces a brown film composition with a high degree of transparency. The film composition of the present invention contains a water-soluble cellulose derivative as a base component, and therefore has excellent strength (impact resistance) even under low-moisture conditions, and also exhibits low values of equilibrium moisture regain. With this property, the film composition of the present invention is useful for a container, particularly a hard capsule-type container, to store substances susceptible to moisture (medicine, food, etc.). The method of the present invention also prevents spot generation or turbidity in coloring a film composition containing a water-soluble cellulose derivative, particularly a capsule base, with caramel, thereby producing a film composition having a high degree of transparency.

### BEST MODE FOR CARRYING OUT THE INVENTION

### I. Brown Film Composition and Preparation Method Thereof.

The brown film composition of the present invention contains a water-soluble cellulose derivative as a film formation material, caramel (Caramel I, II, III or IV) as a brown colorant, and further contains a pH adjuster.

A typical example of the water-soluble cellulose derivative of the present invention may be a cellulose ether containing at least one of an alkyl group or a hydroxy alkyl group as a substituent. The “alkyl group” of the foregoing alkyl group or the hydroxy alkyl group designates a linear or branched C₁₋₆, preferably C₁₋₄, lower alkyl group; more specifically, a methyl group, an ethyl group, a butyl group or a propyl group. Examples of the water-soluble cellulose derivative include lower alkyl cellulose such as methyl cellulose; hydroxy lower alkyl cellulose such as hydroxyethyl cellulose or hydroxy propyl cellulose; and hydroxy lower alkyl alkyl cellulose such as hydroxy ethyl methyl cellulose, hydroxy ethyl ethyl cellulose or hydroxy propyl methyl cellulose. Among these, hydroxy propyl methyl cellulose is particularly preferred because of its film-forming properties and superior mechanical strength under low-moisture conditions.

The water-soluble cellulose derivative used for the present invention preferably ensures that the solution for forming the film or the sheet has a kinetic viscosity of 40 to 40,000 mm²/s. The water-soluble cellulose derivative may be selected from a wide range of commercially-available water-soluble cellulose derivatives as long as it meets this condition, and the selected derivatives may be used solely or in arbitrary combination. Such commercially-available water-soluble cellulose derivatives generally meet a condition that the ratio (Mw/Mn) of the weight average molecular weight (Mw) to the number average molecular weight (Mn) ranges from 1.5 to 4. Note that the weight average molecular weight (Mw) and the number average molecular weight (Mn) for calculating the foregoing ratio (Mw/Mn) may both be found by gel chromatography (size-exclusion chromatography). The theory and the method of the gel chromatography are not particularly limited. A reference for the gel chromatography can be found, for example, in the “Chromatography” chapter, “Size-Exclusion Chromatography” section of “USP30 The United States Pharmacopeia/NF25 The National Formulary”.

The content of the water-soluble cellulose derivative in the brown film composition according to the present invention is not limited, but is generally 70 to 99 wt %, preferably 75 to 99 wt %, more preferably 80 to 99 wt %, and further preferably 85 to 99 wt %, based on 100 wt % of the brown film composition without the moisture content.

Caramel (caramel, caramel color), obtained by heating edible carbohydrates such as sugar or glucose, is a food additive widely used as a brown colorant for food, medicine, cosmetics, feed etc. According to the “List of Existing Food Additives in 1996 Notification No. 120” (the Ministry of Health, Labor and Welfare in Japan), as shown in Table 1, there are four types of caramel: Caramel I, II, III, and IV.

TABLE 1

| | colloidal | |
|---|---|---|
| Name | charge | Definition |
| Caramel I (plain) | weak(−) | A substance obtained by adding acid |
| | | or alkali to, and heat-treating, |
| | | food-grade carbohydrates including |
| | | starch hydrolysates, molasses or |
| | | saccharides, excluding ones |
| | | containing sulfite compounds or |
| | | ammonium compounds. |
| Caramel II (caustic | (−) | A substance obtained by adding |
| sulfite process) | | sulfite compounds (and acid or |
| | | alkali) to, and heat-treating, food- |
| | | grade carbohydrates including starch |
| | | hydrolysates, molasses or |
| | | saccharides, excluding ones |
| | | containing ammonium compounds. |
| Caramel III (ammonia | strong (+) | A substance obtained by adding |
| process) | | ammonium compounds (and acid or |
| | | alkali) to, and heat-treating, food- |
| | | grade carbohydrates including starch |
| | | hydrolysates, molasses or |
| | | saccharides, excluding ones |
| | | containing sulfite compounds. |
| Caramel IV (sulfite | strong | A substance obtained by adding |
| ammonia process) | (−) | sulfite compounds and ammonium |
| | | compounds (and acid or alkali) to, |
| | | and heat-treating, food-grade |
| | | carbohydrates including starch- |
| | | hydrolysates, molasses or |
| | | saccharides. |

Such caramels (Caramels I to IV) can be differentiated from each other using the following identification tests (1) to (3) according to Japanese Standard of Food Additive, Seventh Edition. In particular, the identification test (1), which uses a weak base anion exchanger (DEAE cellulose), and the identification test (2), which uses a strong acid cation exchanger (phosphoryl cellulose), adopts the reaction between the ion-exchange cellulose and the colloidal charge of caramel. By using the reaction with the ion-exchange cellulose thusly, Caramels I to IV can be clearly differentiated from each other.

### Identification Test (1)

A fixed amount of caramel is used to provide approximate absorbence of 0.5. The measured caramel is mixed with 0.025 mol/L hydrochloric acid so that the resulting quantity becomes precisely 100 mL. The liquid is centrifuged as necessary, and the supernatant is removed to be used as “Solution A”. Next, 20 mL of Solution A is mixed with 0.20 g of a weak base diethylaminoethyl-cross-linked cellulose anion exchanger (exchange capacity=0.7 meq/g), and the mixture is well-stirred. After the centrifuge, the supernatant is removed to be used as “Solution B”. Using 0.025 mol/L hydrochloric acid as a control liquid, respective absorbances (X_{A}) and (X_{B}) of Solutions A and B are measured using a cell with a path length of 1 cm at a wavelength of 560 nm, so as to find a value of “(X_{A}−X_{B})/X_{A}”.

### Identification Test (2)

0.20 to 0.30 g of caramel is mixed with 0.025 mol/L hydrochloric acid so that the resulting quantity becomes precisely 100 mL. The liquid is centrifuged as necessary, and the supernatant is removed to be used as “Solution C”. Next, 40 mL of Solution C is mixed with 2.0 g of a strong acid phosphoryl-cross-linked cellulose cation exchanger (exchange capacity=0.85 meq/g), and the mixture is well-stirred. After the centrifuge, the supernatant is removed to be used as “Solution D”. Using 0.025 mol/L hydrochloric acid as a control liquid, respective absorbances (X_{C}) and (X_{D}) of Solutions C and D are measured using a cell with a path length of 1 cm at a wavelength of 560 nm, so as to find a value of “(X_{C}−X_{D})/X_{C}”.

### Identification Test (3)

0.10 g of caramel is mixed with water so that the resulting quantity becomes precisely 100 mL. The liquid is centrifuged as necessary, and the supernatant is removed to be used as “Solution E”. Next, 5 mL of Solution E is mixed with water so that the resulting quantity becomes precisely 100 mL to be used as “Solution F”. Using water as a control liquid, absorbance (X_{E}) of Solution E at a wavelength of 560 nm and absorbance (X_{F}) of Solution F at a wavelength of 280 nm are measured using a cell with a path length of 1 cm, so as to find a value of “X_{F}×20/X_{E}”.

With regard to Caramel I, the measurement value “(X_{A}−X_{B})/X_{A}” of identification test (1) is not more than 0.75, and the measurement value “(X_{C}−X_{D})/X_{C}” of identification test (2) is not more than 0.50. With regard to Caramel II, the measurement value of identification test (1) is not less than 0.50, and the measurement value “X_{F}×20/X_{E}” of identification test (3) is not less than 50. With regard to Caramel III, the measurement value of identification test (1) is not less than 0.50. With regard to Caramel IV, the measurement value of identification test (1) is not less than 0.50, and the measurement value of identification test (3) is not more than 50.

Note that Caramels I, II, III, and IV are all commercially available.

The content of the caramel in the brown film composition of the present invention is not particularly limited. The content is generally determined depending on the target degree of coloring within a range of not more than 15 wt % on the basis of 100 wt % of brown film composition without the moisture content. The content is preferably not more than 13 wt %, more preferably not more than 11 wt %, and further preferably not more than 8 wt %. There is also no specific lower limit of the content; however, a possible lower limit is 1 wt % considering caramel aggregation.

The brown film composition of the present invention is a composition obtained by solidifying the foregoing aqueous solution containing a water-soluble cellulose derivative and caramel into a film or a sheet. The brown film composition of the present invention is characterized in that the pH of the aqueous solution to be formed into a film or a sheet is adjusted by a pH adjuster.

The pH adjuster is appropriately selected according to the colloidal charge of the caramel. More specifically, an acid agent is used as the pH adjuster for Caramel I, II or IV, which have a negative charge, and an alkaline agent is used as the pH adjuster for Caramel III, which has a positive charge.

The acid agent is not limited as long as it is capable of acidifying (lowering the pH of) the aqueous solution containing a water-soluble cellulose derivative and caramel. Examples of the acid agent include inorganic acids and salts thereof, such as hydrochloric acid, perchloric acid, sulfuric acid, nitric acid, phosphoric acid, pyrophosphoric acid, tripolyphosphoric acid, or boric acid; organic acids and salts thereof such as acetic acid, citric acid, fumaric acid, malic acid, phytic acid, adipic acid, gluconic acid, succinic acid, lactic acid, tartaric acid, ascorbic acid, alicylic acid, benzoic acid, oleic acid, myristic acid, maleic acid, glucuronic acid, sorbic acid, dehydroacetic acid, aldaric acid, formic acid, acetic acid, butanoic acid, or propionic acid; amphoteric electrolytes or its salt such as amino acid, especially acidic amino acids such as glutamic acid or aspartic acid. The aldaric acid designates a carboxylic acid obtained by formal oxidation of aldose into an aldehyde group. The acid agent is preferably selected from those usable as a food ingredient. Organic acids such as citric acid are particularly preferable.

In the aqueous solution containing a water-soluble cellulose derivative and caramel thus adjusted in pH using the acid agent, the content of the acid agent is determined to adjust the pH to be lower than that of an aqueous solution (control aqueous solution) identical in composition except for the incorporation of pH adjuster such as the acid agent. As long as this condition is met, the content of the acid agent or the pH value is not particularly limited. For example, when using Caramel I, the pH of the aqueous solution to be formed into a film is generally not more than 7, preferably not more than 6.9, more preferably not more than 6.5, further preferably not more than 6.3, and particularly preferably in a range from pH 6.3 to 4. When using Caramel II, the pH of the aqueous solution to be formed into a film is generally not more than 7, preferably not more than 6.8, more preferably not more than 6.5, further preferably not more than 6.2, and particularly preferably in a range from pH 6.2 to 4. When using Caramel IV, the pH of the aqueous solution to be formed into a film is generally not more than 7, preferably not more than 6.5, more preferably not more than 6.2, further preferably not more than 6, and particularly preferably in a range from pH 6 to 4.

The alkaline agent is not limited as long as it is capable of alkalizing (increasing the pH of) the aqueous solution containing a water-soluble cellulose derivative and caramel. Examples of the alkaline agent include sodium hydroxide, potassium hydroxide, calcium hydroxide, magnesium hydroxide, sodium carbonate, sodium acid carbonate, potassium carbonate, disodium hydrogen phosphate, ammonia, guanidine, imidazole, and amphoteric electrolytes or salts thereof such as amino acid, especially basic amino acids such as arginine, lysine, or histidine.

The alkaline agent is preferably selected from those usable as a food ingredient. Sodium hydroxide, potassium hydroxide, and ammonia are particularly preferable.

In the aqueous solution containing a water-soluble cellulose derivative and caramel thus adjusted in pH using the alkaline agent, the content of the alkaline agent is determined to adjust the pH to be higher than an aqueous solution (control aqueous solution) identical in composition except for the incorporation of pH adjuster such as the alkaline agent. As long as this condition is met, the content of the alkaline agent or the pH value is not particularly limited. The pH of the aqueous solution to be formed into a film is typically not less than 7.6, preferably not less than 7.7, and particularly preferably in a range from pH 7.7 to 9.

As mentioned above, the pH adjuster (acid agent or alkaline agent) may be an amphoteric electrolyte or salt thereof, that includes, in addition to the above-mentioned amino acid and basic amino acid, neutral amino acids such as glycine, alanine, valine, leucine, isoleucine, serine, threonine, cysteine, methionine, phenylalanine, tryptophan, thyrosin, proline, cystine, glutamine, or asparagine. These amphoteric electrolytes serve both as an acid agent to lower the pH of the aqueous solution containing a water-soluble cellulose derivative and caramel, and as an alkaline agent to increase the pH of the aqueous solution containing a water-soluble cellulose derivative and caramel.

In addition to the foregoing components, the brown film composition according to the present invention may contain a gelatinizer. Examples of the gelatinizer include carrageenan, tamarind seed, polysaccharide, pectin, xanthan gum, locust bean gum, curdlan, gelatin, furcelleran agar, and gellant gum. They may be used solely or in arbitrary combination.

Among the above-mentioned gelatinizers, carrageenan has a particularly high gel strength. Carrageenan also increases its gelatinizing property in the presence of a specific ion. In this case, even a small amount of carrageenan exhibits superior gelatinizing property. Considering these advantages, carrageenan is an optimal gelatinizer.

Carrageenan is generally classified into three types: kappa carrageenan, iota carrageenan and lambda carrageenan. Among these, kappa carrageenan and iota carrageenan are preferred in the present invention. Pectin is classified into LM pectin and HM pectin, based on the difference in esterification degree. Gellant gum is also classified into acylation gellant gum (native gelan gum) and deacylation gellant gum according to its acylation property. However, all of those kinds are equally applicable for the present invention.

Depending on the type of gelatinizer, the brown film composition of the present invention may also contain an auxiliary gelatinizer. Examples of auxiliary gelatinizers used with carrageenan as a gelatinizer include, for the kappa-carrageenan, a compound that provides single or plural kinds of potassium ion, ammonium ion and calcium ion, such as potassium chloride, ammonium chloride, acetic acid, ammonium, or calcium chloride; for the iota-carrageenan, a compound that provides calcium ion in water, such as calcium chloride. Examples of auxiliary gelatinizers used with the gellan gum as a gelatinizer includes a compound that provides single or plural kinds of sodium ion, potassium ion, calcium ion and magnesium ion, such as sodium chloride, potassium chloride, calcium chloride, or magnesium sulfate. Organic acids or water-soluble salts thereof, such as citric acids or sodium citrates, are also useful.

When hydroxy propyl methyl cellulose is used as a water-soluble cellulose derivative, carrageenan and potassium chloride are preferred as the gelatinizer and the auxiliary gelatinizer, respectively.

The content of such a gelatinizer in the brown film composition of the present invention is generally 0.05 to 10 wt %, preferably 0.1 to 9.5 wt %, more preferably 0.2 to 9 wt %, and further preferably 0.3 to 8 wt %. The content of an auxiliary gelatinizer such as potassium chloride in the brown film composition is generally not more than 2.2 wt %, preferably 0.1 to 2.1 wt %, more preferably 0.2 to 1.9 wt %, and further preferably 0.3 to 1.6 wt %.

Note that in addition to these essential ingredients, a water-soluble cellulose derivative, caramel and a pH adjuster, the brown film composition of the present invention may also contain a plasticizer, a sequestering agent, opaquer, or perfume.

The plasticizer is not particularly limited as long as it can be used for medicine or food. Examples of the plasticizer include dioctyl adipate, adipic acid polyester, epoxidized soybean oil, epoxy hexahydrophthalic-acid diester, kaolin, triethyl citrate, glycerin, glycerol fatty ester, sesame oil, a mixture of dimethyl polysiloxane and silicon dioxide, D-sorbitol, medium-chain fatty acid triglyceride, corn starch-derived sugar-alcohol liquid, triacetin, concentrated glycerin, castor oil, phytosterol, diethyl phthalate, dioctyl phthalate, dibutyl phthalate, butyl phthalyl butyl glycolate, propylene glycol, polyoxy ethylene (105), polyoxy propylene (5), glycol, polysorbate 80, polyethylene glycol of 400, 600, 1,500, 4,000, or 6,000 average molecular weight (PEG400, PEG600, PEG1500, PEG4000, PEG6000), myristic acid isopropyl, a mixed oil of cottonseed oil and soybean oil, monostearic acid glycerin, and linolic acid isopropyl.

The average molecular weight of PEG can be measured according to “Japanese Pharmacopoeia” and “Japan Pharmaceutical Excipient Standards”, as defined by the Japan Ministry of Health, Labour and Welfare.

### Average Molecular Weight Test

42 g of phthalic anhydride is added to a 1 L lightproof stopper bottle that contains exactly 300 mL of newly-distilled pyridine. After strongly shaking the bottle to dissolve the phthalic anhydride, the bottle is allowed to stand for at least 16 hours. Precisely 25 mL of the resulting liquid is placed into a pressure-proof stopper bottle of about 200 mL(s). Precisely 0.8 to 12.5 g of the PEG sample is added to the pressure-proof stopper bottle. After being sealed and wrapped with a strong cloth, the bottle is placed in a water bath heated to 98±2° C., making sure that the liquid in the bottle is immersed in the liquid in the water bath. After the bottle is kept in the same state for 30 minutes at 98±2° C., it is taken out of the water bath and allowed to cool in the air to room temperature. Next, precisely 0.5 mol/L of water oxidation sodium liquid 50 mL is added to the cooled solution. After further adding five drops of a pyridine solution (1X→100) of phenolphthalein, the resulting liquid is subjected to titration with 0.5 mol/L of the water oxidation sodium liquid. The titration is finished when the liquid is a pale red color for 15 consecutive seconds. A blank experiment is also performed in the same manner.

average molecular weight=(amount of sample(g)×4000)/(a−b) Formula 1

a: consumption (mL) of 0.5 mol/L sodium hydroxide liquid in the blank experiment

b: consumption (mL) of 0.5 mol/L sodium hydroxide liquid in the experiment using the PEG sample.

Note that when a plasticizer is used, the brown film composition (final dry product) of the present invention is generally not more than 15 wt %, preferably not more than 13 wt %, more preferably not more than 11 wt %, and further preferably not more than 8 wt %.

Examples of the sequestering agent include ethylenediamine tetraacetic acids, acetic acids, boric acids, citric acids, gluconic acids, lactic acids, phosphoric acids, tartaric acids, their salts, meta-phosphate, dihydroxyethyl glycine, lecitin, β-cyclodextrin. They may be used solely or in combination.

The opaquer and perfume are not particularly limited as long as they can be used for medicine or food.

The brown film composition of the present invention is typically produced by dissolving the foregoing ingredients, particularly the essential ingredients, the water-soluble cellulose derivative, the caramel and the pH adjuster, in water, extending (flow casting) the liquid into a film or a sheet, and solidifying the film or the sheet (casting method). Also, the brown film composition of the present invention may be produced by a conventional film/sheet formation method, such as the calendar method, the extrusion method, the flat-die method, or the inflation method.

The brown film composition of the present invention may be formed, for example, in the following manner. A water-soluble cellulose derivative is dispersed in water at approximately 70 to 80° C. The liquid is then cooled (generally to 35 to 60° C., more preferably 40 to 60° C.). Another aqueous solution in which caramel and a pH adjuster are dissolved is added to the cooled jellylike liquid in which the water-soluble cellulose derivative is dissolved, and they are evenly mixed. The aqueous solution thus adjusted to the desired pH according to the type of caramel is extended (flow-casted) on a flat plate in the form of a film or a sheet. The film or sheet is then dried to a solid. Any method can be used for the drying process to remove the solvent, including natural drying, air-drying or heated-air drying.

The brown film composition can also be formed to a desired thickness more easily by gelatinizing the aforementioned pH-adjusted, mixed aqueous solution. This is based on the principle that the water-soluble cellulose derivative is gelatinized at a temperature of 60° C. or greater. The process is performed as follows. The mixed aqueous solution is extended (flow-casted) on a flat plate, which is heated to 60° C. or greater, in the form of a film or a sheet so that the solution is gelatinized and dried to a solid. Note that, in the case of a mixed aqueous solution containing a gelatinizer and an auxiliary gelatinizer, the aqueous solution is gelatinized by cooling. Therefore, in this case, the aqueous solution is extended (flow-casted) on a cooled flat plate, or the solution is cooled by air drying after being formed into a film or a sheet on a plate so that the film or sheet is dried to a solid.

The thickness of the film can be adjusted when extending the solution in the form of film or sheet during this production process. The thickness of the film composition of the present invention is generally not less than 10 μm, preferably 20 to 2,000 μm, and more preferably 50 to 500 μm.

The proportions of the respective components in the film-forming aqueous solution are preferably determined to ensure that the kinetic viscosity (20±0.1° C.) of the film-forming aqueous solution ranges from 40 to 40,000 mm²/s. The kinetic viscosity of the film-forming aqueous solution preferably ranges from 90 to 22,000 mm²/s, more preferably from 350 to 22,000 mm²/s, and further preferably 5,000 to 15,000 mm²/s. Note that the kinetic viscosity of the present invention can be measured according to the method of Reference Example 1.

The proportions are not limited as long as they fall within the foregoing range; however, the content of the water-soluble cellulose derivative generally ranges from 1 to 60 wt %, preferably from 5 to 50 wt %, and more preferably from 10 to 30 wt %; and the content of the caramel generally ranges from 0.2 to 10 wt %, preferably from 0.2 to 8 wt %, more preferably from 0.3 to 8 wt %, and further preferably from 0.3 to 5 wt %.

The proportion of the pH adjuster in the film-forming aqueous solution is adjusted appropriately according to the following reference range. The content of the pH adjuster (particularly for an acid agent) when Caramel I is used is determined so that the pH becomes lower than that of an aqueous solution (control aqueous solution) identical in composition except for the incorporation of the pH adjuster. More specifically, the content is determined so that the pH of the aqueous solution is not more than 7, preferably not more than 6.9, more preferably not more than 6.5, further preferably not more than 6.3, and particularly preferably in a range from pH 6.3 to 4. Similarly, when Caramels II or IV is used, the content is determined so that the pH becomes lower than that of an aqueous solution (control aqueous solution) identical in composition except for the incorporation of the pH adjuster. The content of the pH adjuster in the case of using Caramel II is determined so that the pH of the film-forming aqueous solution becomes not more than 7, preferably not more than 6.8, more preferably not more than 6.5, further preferably not more than 6.2, and particularly preferably in a range from 6.2 to 4. When Caramel IV is used, the content of the pH adjuster is determined so that the pH of the film-forming aqueous solution becomes not more than 7, preferably not more than 6.5, more preferably not more than 6.2, further preferably not more than 6, and particularly preferably in a range from pH 6 to 4.

When Caramel III is used, the content of the pH adjuster (mostly alkaline agent) is determined so that the pH is set higher than that of an aqueous solution (control aqueous solution) identical in composition except for the incorporation of the pH adjuster. The content is determined so that the pH of the film-forming aqueous solution is generally not less than 7.6, preferably not less than 7.7, and particularly preferably in a range from 7.7 to 9.

When a gelatinizer is used, its content in the film-forming aqueous solution is generally 0.01 to 0.5 wt %, preferably 0.02 to 0.45 wt %, and more preferably 0.03 to 0.4 wt %. When an auxiliary gelatinizer is used, its content in the film-forming aqueous solution is generally 0.01 to 0.5 wt %, preferably 0.02 to 0.45 wt %, and more preferably 0.03 to 0.4 wt %.

### II. Container, Especially a Capsule-Type Container, and Capsule Formulation

The brown film composition of the present invention prepared in the above-described manner has excellent strength (impact resistance) even in low-moisture conditions, and also exhibits low values of equilibrium moisture regain. With this characteristic, the brown film composition of the present invention is useful as a container for storing substances susceptible to moisture (medicine, food, etc.). The brown film composition of the present invention is particularly useful for a hard capsule-type container. The present invention thus provides a container, especially a hard capsule-type container, totally or partially comprising the above-mentioned brown film composition. The capsule-type container of the present invention can be manufactured using a common immersion method, specifically as follows. A capsule-forming pin is dipped in a film-forming aqueous solution (hereinafter referred to as “capsule base solution” or “base solution”) containing the foregoing ingredients. After the pin is pulled out of the solution, the layer of the base solution formed around the outer dimension of the capsule-forming pin is cooled to be gelatinized.

Note that the contents of ingredients in the capsule base solution are adjustable by appropriately changing the proportions of the ingredients in the film-forming aqueous solution.

The water content of the water in the capsule base solution is not limited; however, the viscosity of the capsule base solution is generally 100 to 20,000 m Pa·s, preferably 300 to 10,000 m Pa·s, and further preferably 1,000 to 50,000 m Pa·s under the temperature (temperature of immersion liquid) in the immersion process for the capsule-forming pin (30 to 80° C., preferably 40 to 60° C.). More specifically, the water content is generally 60 to 90 wt %, and preferably 70 to 85 wt %.

The method for preparing the capsule base solution (immersion liquid) is not particularly limited. In one exemplary method, a water-soluble cellulose derivative is dispersed in purified water heated to approximately 70 to 80° C. in which a gelatinizer and/or an auxiliary gelatinizer is dissolved as necessary. The dispersion is then cooled to a desired temperature to obtain the target immersion liquid (generally 35 to 60° C., more preferably 40 to 60° C.). The resulting jellylike liquid is mixed evenly with an aqueous solution containing caramel and a pH adjuster, so as to prepare an uniform brown solution. In another exemplary method, a water-soluble cellulose derivative is dispersed in water heated to approximately 70 to 80° C., and the solution is cooled to dissolve the water-soluble cellulose derivative. After that, a gelatinizer and/or an auxiliary gelatinizer is dissolved in the solution as necessary. The resulting solution is heated again to approximately 30 to 50° C., and mixed with an aqueous solution containing caramel and a pH adjuster, so as to prepare a uniform brown solution. The temperature of the brown solution is adjusted to obtain the target immersion liquid.

The capsule-type container of the present invention is produced by first dipping the capsule-forming pin in the capsule base solution (immersion liquid), then pulling the capsule-forming pin out of the capsule base solution, after which the layer of the base solution formed around the outer dimension of the capsule-forming pin is cooled to be gelatinized. Thereafter, the gel film is dried at 20 to 80° C. More specifically, the capsule-type container of the present invention is manufactured through the following steps.

(1) Step of dipping a capsule-forming pin in a capsule base solution (immersion liquid) containing a water-soluble cellulose derivative, caramel and a pH adjuster (and a gelatinizer and an auxiliary gelatinizer as necessary) (dipping step).

(2) Step of pulling the capsule-forming pin out of the capsule base solution, and gelatinizing the layer of the base solution formed around the outer dimension of the capsule-forming pin (gelatinization step).

(3) Step of drying the capsule base formed as a gel layer on the outer portion of the capsule-forming pin (drying step).

(4) Step of removing the dry capsule base from the capsule-forming pin (removal step).

The following heating process (5) may be performed after the gelatinization step (2), before or during the drying process (3), or after the removal step (4).

(5) Step of heating the gel capsule base at 50 to 150° C. (heating process).

When a capsule base solution (immersion liquid) not containing a gelatinizer such as carrageenan is used, step (2) can be performed by using a capsule-forming pin heated to 60° C. or higher (thermal gelatinization), which relies on the fact that the water-soluble cellulose derivative is gellatinized at a temperature equal to or more than 60° C. Specifically, the thermal gelatinization is performed as follows. A capsule-forming pin heated to an appropriate temperature according to the liquid temperature, for example, 60 to 150° C., preferably 60 to 120° C., and more preferably 70 to 90° C., is dipped in a capsule base solution (immersion liquid) whose temperature is kept constant at 35 to 50° C., preferably 35 to 45° C. Then, in gelatinization step (2), the capsule-forming pin is pulled out of the capsule base solution (immersion liquid), so that the capsule base solution formed around the outer dimension of the capsule-forming pin is gelatinized.

Meanwhile, when a capsule base solution (immersion liquid) containing a gelatinizer such as carrageenan is used, step (2) can be performed by adjusting the temperature in the vicinity of the capsule manufacturing apparatus to generally not more than 35° C., preferably not more than 30° C., and more preferably not more than room temperature, so as to cool the capsule base solution formed around the outer dimension of the capsule-forming pin (cooling gelatinization). This relies on the fact that the solution is gellatinized at a temperature of 50° C. or lower. Specifically, the cooling gelatinization is performed as follows. In step (1), a capsule-forming pin heated to an appropriate temperature according to the liquid temperature, for example, 10 to 30° C., preferably 13 to 28° C., and more preferably 15 to 25° C., is dipped in a capsule base solution (immersion liquid) whose temperature is kept constant at 35 to 50° C., preferably 40 to 60° C. Then, in gelatinization step (2), the capsule-forming pin is pulled out of the capsule base solution (immersion liquid), so that the capsule base solution formed around the outer dimension of the capsule-forming pin is gelatinized.

The drying process (3) can be performed at room temperature, often by blowing air of room temperature. The removal step (4) is carried out by removing the dry capsule base, formed around the outer portion of the capsule-forming pin, from the capsule-forming pin.

The arbitrary heating process (5) can be performed after gelatinization step (2)—in other words, after the capsule base solution is gelatinized (solidified). Heating may be performed any time after gelatinization step (2), for example, before, after, or during the drying process (3), or after the removal process (4). However, heating is preferably performed as follows. After gelatinization step (2), the gel capsule base is subjected to the drying process under room temperature. When the gel capsule base is dried or half-dried, the capsule base is heated. The heating temperature is not particularly limited; however, it preferably ranges from 60 to 100° C., more preferably from 60 to 80° C. Generally, the heating can be performed by sending air of 50 to 150° C.

The capsule base thus prepared is cut to a predetermined length, and becomes available as a capsule body and cap set, or as separate parts. The capsule-type container of the present invention designates a product that contains, at least either in the capsule body or in the cap, a brown capsule base prepared by the foregoing method. In other words, the scope of the present invention includes both a product comprising the brown capsule base either in the body or in the cap, and a product comprising the brown capsule base both in the body and in the cap.

The capsule base of the present invention prepared in the foregoing manner contains a water-soluble cellulose derivative in an amount of 70 to 99 wt %, preferably 75 to 99 wt %, more preferably 80 to 99 wt %, and further preferably 85 to 99 wt %. The content of the caramel is appropriately determined according to the desired coloring degree. The content is generally not more than 15 wt %, preferably not more than 13 wt %, more preferably not more than 11 wt %, and further preferably not more than 8 wt %. The content needs to be at least 0.1 wt % (lower limit) to ensure the target coloring; however, the lower limit is preferably 1 wt % in consideration of caramel aggregation.

Further, when the capsule base of the present invention contains a gelatinizer, the content is generally 0.05 to 10 wt %, preferably 0.1 to 9.5 wt %, more preferably 0.2 to 9 wt %, and further preferably 0.3 to 8 wt %. When the capsule base of the present invention contains an auxiliary gelatinizer, the content is not more than 2.2, preferably 0.1 to 2.1 wt %, more preferably 0.2 to 1.9 wt %, and further preferably 0.3 to 1.6 wt %. When the capsule base of the present invention contains a plasticizer, the content is generally not more than 15 wt %, preferably not more than 13 wt %, more preferably not more than 11 wt %, further preferably not more than 8 wt %.

The capsule-type container comprising the capsule base according to the present invention can be filled with medicine, food, cosmetics, agrichemicals or feed. By containing these fillings, the capsule-type container is completed as a capsule formulation.

The range of filling is not limited as long as it does not dissolve or react with the capsule film of the present invention. Typical fillings are solid materials such as powders or granules; however, various liquids or gels may also be contained in the capsule. Suitable liquids include alcohols such as stearyl alcohol, cetanol, polyethylene glycol (those of 600, 800, 1,000, 1,500, 2,000, 3,000, 4,000, 6,000, 8,000 and 20,000 average molecular weight); oils such as sesame oil, soybean oil, arachis oil, corn oil, hydrogenated oil, paraffin oil; fatty acids such as white beeswax, stearic acid, palmitic acid, myristic acid, triethyl citrate, triacetone, medium-chain triglyceride; and their derivatives. These liquid substances are usually mixed with active ingredients or the main components of medicine, food, or cosmetics in the capsule base of the present invention.

The type of drug to be contained in the capsule base of the present invention is not limited. Typical examples are oral administration drugs such as vitamins, antifebriles, painkillers, antiphlogistics, anti-tumor agents, cardiotonics, anticoagulants, hemostats, osteoclastic inhibitors, vascularization inhibitors, antidepressants, antiulcer drugs such as proton pump inhibitors including benzimidazole derivatives, expectorants/cough suppressants, antiepileptic agents, antiallergic agents, antiarrhythmics, vasodepressors, hypotensive diuretics, diabetic medicine, antituberculous agents, hormone drugs, antinarcotics, and many more oral drugs.

Restoration of such fillings into the capsule of the present invention may be performed using a publicly known capsule filling machine, such as a fully automatic capsule filling machine, or a capsule filling/sealing machine. An example of a fully automatic capsule filling machine is a Qualicaps (Model Number: LIQFIL super 80/150) fully automatic capsule filling machine. An example of a capsule filling/sealing machine is a Qualicaps (Model Number: LIQFIL super FS) capsule filling/sealing machine.

As described above, the capsule-type container of the present invention is made of a film having a low moisture content, and exhibits low values of equilibrium moisture regain. This allows the capsule-type container of the present invention to be filled with a component that reacts relatively easily with moisture (e.g. a component that easily changes in quality by moisture) as a capsule formulation. Examples of the component susceptible to moisture include ester compounds and enzyme. Further, the capsule-type container of the present invention has excellent strength (impact resistance), even in low-moisture conditions. Because of this property, the capsule-type container of the present invention can be stored under dry conditions when it contains a highly hygroscopic component or a component that reacts relatively easily with moisture, thereby protecting the component from change in quality. Further, the capsule-type container of the present invention is made of a film having low hygroscopicity, and is therefore capable of containing a component having a relatively high moisture content such as a substance containing a large amount of hydrated water, including enzymes and morphine.

### III. A Method for Suppressing the Generation of Caramel Aggregate in the Brown Film Composition

The present invention provides a method for suppressing the generation of caramel aggregate in the brown film composition prepared by using a water-soluble cellulose derivative and caramel as essential ingredients.

As for the water-soluble cellulose derivative and the caramel, the same substances and proportions as those listed above in section I can be adopted. Among the listed water-soluble cellulose derivatives, hydroxy propyl methyl cellulose is particularly preferred.

To attain the intended suppression, the present method incorporates a pH adjuster in the aqueous solution (film-forming aqueous solution) containing a water-soluble cellulose derivative and caramel for preparing the brown film composition, so that the pH of the aqueous solution falls within a desired range according to the type of caramel.

More specifically, when Caramels I, II or IV are used, a pH adjuster (preferably an acid agent) is added to the film-forming aqueous solution so that the pH of the forming aqueous solution becomes lower than that of an aqueous solution (control aqueous solution) identical in composition except for the incorporation of the pH adjuster. When Caramel I is used, the pH is preferably not more than 7, preferably not more than 6.9, more preferably not more than 6.5, further preferably not more than 6.3, and particularly preferably in a range from pH 6.3 to 4. When Caramel II is used, the pH is preferably not more than 7, preferably not more than 6.8, more preferably not more than 6.5, further preferably not more than 6.2, and particularly preferably in a range from pH 6.2 to 4. When Caramel IV is used, the pH is not more than 7, preferably not more than 6.5, more preferably not more than 6.2, further preferably not more than 6, and particularly preferably in a range from pH 6 to 4.

Further, when Caramel III is used, a pH adjuster (preferably an alkaline agent) is added to the film-forming aqueous solution so that the pH of the forming aqueous solution becomes higher than that of an aqueous solution (control aqueous solution) identical in composition except for the incorporation of the pH adjuster. In this case, the pH is not less than 7.6, preferably not less than 7.7, and particularly preferably in a range from pH 7.7 to 9.

By thus using a pH adjuster in addition to the water-soluble cellulose derivative and the caramel, aggregation of caramel is suppressed. Because of this effect, it is possible to produce a clear brown film composition with a high degree of transparency. The preparation of the brown film composition is performed according to the method described above in section II. As also described in the section II, the brown film composition obtained by the method is useful for a capsule base or a container for various medicine, food, cosmetics, agrichemicals, feed etc.

### EXAMPLES

The following will describe the present invention by way of Experiment Examples and Examples. It should be noted, however, that the present invention is in no way limited by the following descriptions. Unless otherwise specified, the “percentage” used below means percent by weight.

### Reference Example 1

A certain amount of hydroxypropyl methylcellulose (HPMC) (Shin-Etsu Chemical Co., Ltd.; weight average molecular weight of 60,000, Mw/Mn=1.9 determined here and below by gel chromatography measurement) that had been measured to give the predetermined content (3 to 18 wt %) shown in Table 3 was placed in a jar, and calcium chloride was added to make a final concentration of 2%. The quantity of the mixture was adjusted to 500 g by adding hot water.

The container was capped and the mixture was stirred with a stirrer at 350 to 450 rpm for 10 to 20 minutes until a uniform dispersion liquid was obtained. The viscosity of the resulting liquid was measured by stirring the mixture and dissolving its contents for 20 to 40 minutes in a water bath set to a temperature of 10° C. or less. Measurement was made at 20±0.1° C. by a method using a single cylindrical rotation viscometer (Brookfield-type viscometer, model LV), under the following conditions.

TABLE 2
Measurement Conditions

| | | | Conversion |
|---|---|---|---|
| Viscosity (mPa · s) | Cylinder Number | RPM | multiplier |
| 600 or greater to less than 1,400 | 3 | 60 | 20 |
| 1,400 or greater to less than | 3 | 12 | 100 |
| 3,500 | | | |
| 3,500 or greater to less than | 4 | 60 | 100 |
| 9,500 | | | |
| 9,500 or greater to less than | 4 | 6 | 1,000 |
| 99,500 | | | |
| 99,500 or greater | 4 | 3 | 2,000 |

### Apparatus Operation

The single cylindrical rotation viscometer was activated to rotate for two minutes, and the operation was suspended for two minutes after recording the reading on the meter. This procedure was repeated and a total of three readings (absolute viscosity: mPa·s) was averaged.

The solutions were degassed under reduced pressure and allowed to stand for 12 hours at room temperature to obtain clear HPMC jellies of varying concentrations. The density of each HPMC jelly (mass/volume) was measured at 20±0.1° C. Using a device to prepare a thin-layer plate for thin-layer chromatography, the HPMC jelly was cast over a glass plate to prepare a thin HPMC jelly film, which was then dried for one hour at 60° C. to 100° C. to obtain a film about 120 μm thick.

Table 3 below shows kinetic viscosities (absolute viscosity/density: mm²/s) of the respective HPMC jellies, along with the results of the evaluation of ease of film formation (operability).

TABLE 3

| HPMC content in | Kinematic viscosity of | Ease of film formation |
|---|---|---|
| HPMC jelly | HPMC jelly (mm²/s) | (operability) |
| 3% | 40 | Poor |
| 4% | 90 | Good |
| 6% | 350 | Good |
| 8% | 1,000 | Good |
| 10% | 2,500 | Good |
| 12% | 5,000 | Excellent |
| 15% | 15,000 | Excellent |
| 16% | 22,000 | Good |
| 18% | 40,000 | Average |

Ease of film formation (operability)
Excellent: Desirable formation
Good: Film formation possible
Average: Film formation possible, but degassing takes a long time in jelly preparation
Poor: Film formation difficult due to low viscosity, but is possible using a mold or spraying method

From the results of the evaluation of ease of film formation (operability in film formation), a desirable range of viscosity for film formation was found to be 40 to 40,000 mm²/s, more preferably 90 to 22,000 mm²/s, further preferably 350 to 40,000 mm²/s, and particularly preferably 5,000 to 15,000 mm²/s.

### Experiment Example 1

In this experiment example, the following experiment was conducted using Caramel I as an example of a caramel having a negative colloidal charge.

200 g of hydroxypropyl methylcellulose (HPMC) (Shin-Etsu Chemical Co., Ltd.) was added and suspended in 800 g of purified water heated to 80° C. The suspension was degassed under reduced pressure and cooled to 55° C. to prepare a HPMC jelly. Separately, 12 g of Caramel I (caramel SF-31; Ikedatohka Industries Co., Ltd.) was dissolved in citric acid aqueous solutions of varying concentrations (Examples 1 to 5) and the aqueous solution of pH 1.2 (Example 6) shown in Table 1, so as to prepare 15% caramel aqueous solutions. Note that the aqueous solution of pH 1.2 is equivalent to the first solution (artificial gastric fluid) specified in the disintegration test of the Japanese Pharmacopoeia, Thirteenth Edition, and it is prepared from 1,000 mL of an aqueous solution containing 2 g of sodium chloride and 7 mL of hydrochloric acid, adjusting the pH to about 1.2.

Each 15% caramel aqueous solution was added to the HPMC jelly (20% HPMC aqueous solution) kept at 55° C., using a three-one motor (HEIDON) to stir the mixture. As a result, a uniform brown jelly was obtained. The brown jelly, stirred continuously at 30 rpm with a three-one motor, was sampled immediately after preparation, and at 2 hours, 6 hours, 12 hours, 24 hours, and 96 hours post-preparation. From each sample, a film about 100 μm thick was prepared. The film was observed with a magnifier to visually check for a brown aggregate (caramel aggregate).

As a comparative example, a 15% caramel aqueous solution (pH 5.8) prepared by dissolving 12 g of Caramel I in purified water was added to the HPMC jelly (20% HPMC aqueous solution) to obtain a uniform brown jelly as above, and the jelly was sampled over time to check for a brown aggregate (caramel aggregate) in the film.

Table 4 shows the pH of the caramel aqueous solution and the pH of the HPMC jelly after the addition of the caramel aqueous solution. Table 5 shows the results of the experiment.

TABLE 4
pH of 15% caramel solution and HPMC jelly

| | | | pH of HPMC |
|---|---|---|---|
| | | pH of 15% | jelly with |
| | | caramel aqueous | caramel aqueous |
| | Dissolving liquid | solution | solution |
| Examples | of Caramel I | (24° C.) | (55° C.) |
| Comparative | Purified water | 5.80 | 7.22 |
| Example 1 | | | |
| Example 1 | 0.01% citric acid | 5.58 | 6.90 |
| | aqueous solution | | |
| Example 2 | 0.02% citric acid | 5.36 | 6.75 |
| | aqueous solution | | |
| Example 3 | 0.05% citric acid | 5.06 | 6.64 |
| | aqueous solution | | |
| Example 4 | 0.1% citric acid | 4.87 | 6.28 |
| | aqueous solution | | |
| Example 5 | 0.2% citric acid | 4.36 | 5.82 |
| | aqueous solution | | |
| Example 6 | aqueous solution, | 2.18 | 3.27 |
| | pH 1.2 | | |

TABLE 5

| | Time after preparation | | | | | |
|---|---|---|---|---|---|---|
| | Immediately | | | | | |
| | after | 2 | 6 | 12 | 24 | 96 |
| | preparation | hours | hours | hours | hours | hours |
| Comparative | − | + | ++ | ++ | ++ | ++ |
| Example 1 | | | | | | |
| (purified water) | | | | | | |
| Example 1 | − | − | − | − | − | + |
| (0.01% citric acid | | | | | | |
| aqueous solution) | | | | | | |
| Example 2 | − | − | − | − | − | + |
| (0.02% citric acid | | | | | | |
| aqueous solution) | | | | | | |
| Example 3 | − | − | − | − | − | + |
| (0.05% citric acid | | | | | | |
| aqueous solution) | | | | | | |
| Example 4 | − | − | − | − | − | − |
| (0.1% citric acid | | | | | | |
| aqueous solution) | | | | | | |
| Example 5 | − | − | − | − | − | − |
| (0.2% citric acid | | | | | | |
| aqueous solution) | | | | | | |
| Example 6 | − | − | − | − | − | − |
| (aqueous solution, | | | | | | |
| pH 1.2) | | | | | | |

++: Aggregation, +: Little aggregation, −: No aggregation

As shown in Table 5, formation of caramel aggregate was observed two hours after preparation in the HPMC jelly (Comparative Example 1) prepared from the caramel aqueous solution using purified water. In contrast, no precipitation of aggregate was observed for extended periods of time after preparation in the HPMC jellies (Examples 1 to 6) that were prepared from the caramel solution using the citric acid aqueous solution or the aqueous solution of pH 1.2, and particularly in the HPMC jellies (Examples 4 to 6) that were prepared from the citric acid aqueous solution of 0.1% or greater concentrations, or the aqueous solution of pH 1.2.

### Experiment Example 2

In this experiment example, the following experiment was conducted using Caramel III as an example of a caramel having a positive colloidal charge.

200 g of hydroxypropyl methylcellulose (HPMC) (Shin-Etsu Chemical Co., Ltd.) was added and suspended in 800 g of purified water heated to 80° C. The suspension was degassed under reduced pressure and cooled to 55° C. to prepare a HPMC jelly (20% HPMC aqueous solution). Separately, a 15% aqueous solution of Caramel III (caramel LF-141, Ikedatohka Industries Co., Ltd.) was adjusted to pH 8 or greater using a 10% sodium hydroxide aqueous solution. The 15% caramel aqueous solution was added to the HPMC jelly (20% HPMC aqueous solution) kept at 55° C., using a three-one motor (HEIDON) to stir the mixture. As a result, a uniform brown jelly was obtained (Examples 7 and 8).

The brown jelly, stirred continuously at 30 rpm with a three-one motor, was sampled immediately after preparation, and at 2 hours, 6 hours, 12 hours, 24 hours, and 96 hours post-preparation. From each sample, a film about 100 μm thick was prepared. The film was observed with a magnifier to visually check for a brown aggregate (caramel aggregate).

As a comparative example, a 15% caramel aqueous solution prepared by dissolving 12 g of Caramel III in purified water, a 0.1% citric acid aqueous solution, or a 0.5% citric acid aqueous solution was added to the HPMC jelly to obtain a uniform brown jelly as above (Comparative Examples 2 to 4), and the jelly was sampled over time to check for a brown aggregate (caramel aggregate) in the film.

Table 6 shows the pH of the caramel aqueous solution and the pH of the HPMC jelly after the addition of the caramel aqueous solution. Table 7 shows the results of the experiment.

TABLE 6

| | | | pH of HPMC |
|---|---|---|---|
| | | pH of 15% | jelly with |
| | | caramel aqueous | caramel aqueous |
| | Dissolving liquid | solution | solution |
| Examples | of Caramel III | (24° C.) | (55° C.) |
| Comparative | Purified water | 6.01 | 7.30 |
| Example 2 | | | |
| Comparative | 0.1% citric acid | 5.47 | 6.90 |
| Example 3 | aqueous solution | | |
| Comparative | 0.5% citric acid | 4.46 | 6.15 |
| Example 4 | aqueous solution | | |
| Example 7 | NaOH aqueous | 8.10 | 7.70 |
| | solution | | |
| Example 8 | NaOH aqueous | 8.99 | 7.95 |
| | solution | | |

TABLE 7

| | Time after preparation | | | | | |
|---|---|---|---|---|---|---|
| | Immediately | | | | | |
| | after | 2 | 6 | 12 | 24 | 96 |
| | preparation | hours | hours | hours | hours | hours |
| Comparative | − | + | ++ | ++ | ++ | ++ |
| Example 2 | | | | | | |
| Comparative | + | ++ | ++ | ++ | ++ | ++ |
| Example 3 | | | | | | |
| (0.1% citric acid | | | | | | |
| aqueous solution) | | | | | | |
| Comparative | ++ | ++ | ++ | ++ | ++ | ++ |
| Example 4 | | | | | | |
| (0.5% citric acid | | | | | | |
| aqueous solution) | | | | | | |
| Example 7 | − | − | − | − | − | − |
| (NaOH aqueous | | | | | | |
| solution) | | | | | | |
| Example 8 | − | − | − | − | − | − |
| (NaOH aqueous | | | | | | |
| solution) | | | | | | |

++: Aggregation, +: Little aggregation, −: No aggregation

As shown in Table 7, formation of caramel aggregate was observed two hours after preparation in the HPMC jelly (Comparative Example 2) prepared from the Caramel III aqueous solution using purified water. In contrast, there was marked suppression of aggregate formation in samples with the increased pH in the caramel aqueous solution and the HPMC jelly (Examples 7 and 8). In samples with the low pH caramel aqueous solutions, aggregation occurred immediately after preparation, as shown in Comparative Examples 3 and 4.

### Experiment Example 3

4 g of potassium chloride and 4 g of carrageenan were dissolved in 632 g of purified water kept at about 70° C. Then, 200 g of hydroxypropyl methylcellulose was dispersed in the solution by stirring. The temperature of the dispersion liquid was lowered to 55° C. and the hydroxypropyl methylcellulose was dissolved therein by stirring. Then, 160 g of a Caramel I aqueous solution prepared using a 0.1% citric acid aqueous solution (0.1% citric acid aqueous solutions containing varying concentrations (0.5% to 60%) of Caramel I) was added by stirring. The mixture was degassed under reduced pressure to prepare a capsule base aqueous solution. The aqueous solution (capsule base aqueous solution) was used as an immersion liquid by charging it into a common capsule manufacturing apparatus employing an immersion method, and hard, brown capsules (Size 1) were prepared according to ordinary methods, with the temperature of the immersion liquid maintained at 50° C. to 52° C. (Examples 9 to 18).

As comparative examples, Caramel I aqueous solutions of varying concentrations (0.5% to 60%) were prepared using purified water instead of a 0.1% citric acid aqueous solution. Using these Caramel I aqueous solutions as pigments, hard, brown capsules were prepared as above (Comparative Examples 5 to 14).

The hard capsules were visually checked for a brown aggregate (caramel aggregate), and capsule strength was measured according to the method below.

### Measurement of Capsule Strength

Using an impact resistance tester (Qualicaps Co., Ltd.), a 50 g weight held from a 10 cm height was released to hit a blank capsule (20 blank capsules from each sample) after free fall. Capsule strength was evaluated according to the percentage of cracked or deformed capsules.

The results are shown in Table 8.

TABLE 8

| | | Caramel | | | |
|---|---|---|---|---|---|
| | Concentration of | concentration in | | Formation of | Capsule |
| Examples | Caramel I solution | base solution | Capsule color | Aggregate | strength |
| Example 9 | 0.5% | 0.08% | Pale yellow | − | Good |
| Comparative | | | Pale yellow | − | Good |
| Example 5 | | | | | |
| Example 10 | 1% | 0.16% | Pale brown | − | Good |
| Comparative | | | Pale brown | − | Good |
| Example 6 | | | | | |
| Example 11 | 2% | 0.32% | Light brown | − | Good |
| Comparative | | | Light brown | + | Good |
| Example 7 | | | | | |
| Example 12 | 5% | 0.8% | Light brown | − | Good |
| Comparative | | | Light brown | ++ | Good |
| Example 8 | | | | | |
| Example 13 | 10% | 1.6% | Brown | − | Good |
| Comparative | | | Brown | ++ | Good |
| Example 9 | | | | | |
| Example 14 | 15% | 2.4% | Brown | − | Good |
| Comparative | | | Brown | ++ | Good |
| Example 10 | | | | | |
| Example 15 | 20% | 3.2% | Brown | − | Good |
| Comparative | | | Brown | ++ | Good |
| Example 11 | | | | | |
| Example 16 | 30% | 4.8% | Brown | − | Good |
| Comparative | | | Brown | + | Good |
| Example 12 | | | | | |
| Example 17 | 50% | 8% | Dark brown | − | Good |
| Comparative | | | Dark brown | ± | Good |
| Example 13 | | | | | |
| Example 18 | 60% | 9.6% | Dark brown | − | Average |
| Comparative | | | Dark brown | ± | Average |
| Example 14 | | | | | |

Formation of Aggregate
++: Marked aggregation
+: Aggregation
±: Little aggregation
−: No aggregation
Capsule Strength
Good: No cracking or deformation in all 20 capsules
Average: Cracking or deformation in 1 to 2 capsules out of 20 capsules

As shown in Table 8, light to dark brown aggregates were observed in the capsule base of the hard capsules in Comparative Examples 7 to 14. This is in contrast to the analogous hard capsules of Examples 11 to 18, in which the formation of aggregates was markedly suppressed. The hard capsules of the Examples were all brown in color, and had excellent transparency and glossiness.

### Experiment Example 4

200 g of hydroxypropyl methylcellulose (Shin-Etsu Chemical Co., Ltd.) was dispersed by stirring in 640 g of purified water kept at about 70° C. The dispersion liquid was cooled to 25° C. and hydroxypropyl methylcellulose was dissolved therein by stirring. Then, 160 g of an aqueous solution containing 40% methylcellulose and Caramel I (caramel SF-31, Ikedatohka Industries Co., Ltd.) that had been adjusted with a 0.1% citric acid aqueous solution (0.1% citric acid aqueous solutions containing varying concentrations—from 0.5% to 60%—of Caramel I) was added by stirring. The mixture was allowed to stand for seven hours for degassing. The resulting aqueous solution (capsule base aqueous solution) was used as an immersion liquid by charging it into a common capsule manufacturing apparatus using an immersion method, and the temperature of the immersion liquid was maintained at 39° C. to 41° C. The immersion liquid was then applied to a capsule mold heated to 80° C. and dried at 60° C. to prepare hard, brown capsules (Size 1; Examples 19 to 28).

As comparative examples, aqueous solutions containing 40% methyl cellulose and varying concentrations (0.5% to 60%) of Caramel I that had been adjusted with purified water instead of 0.1% citric acid aqueous solution were prepared. Using these aqueous solutions as pigments, hard, brown capsules were prepared as above (Comparative Examples 15 to 24).

The hard capsules were visually checked for a brown aggregate (caramel aggregate) as in Experiment Example 3. The results of the inspection are shown in Table 9, along with the results of the measurement of capsule strength.

TABLE 9

| | Concentration | Caramel | | | |
|---|---|---|---|---|---|
| | of Caramel I | concentration | | Formation of | Capsule |
| Examples | solution | in jelly | Capsule color | Aggregate | strength |
| Example 19 | 0.5% | 0.08% | Pale yellow | − | Good |
| Comparative | | | Pale yellow | − | Good |
| Example 15 | | | | | |
| Example 20 | 1% | 0.16% | Pale brown | − | Good |
| Comparative | | | Pale brown | − | Good |
| Example 16 | | | | | |
| Example 21 | 2% | 0.32% | Light brown | − | Good |
| Comparative | | | Light brown | + | Good |
| Example 17 | | | | | |
| Example 22 | 5% | 0.8% | Light brown | − | Good |
| Comparative | | | Light brown | ++ | Good |
| Example 18 | | | | | |
| Example 23 | 10% | 1.6% | Brown | − | Good |
| Comparative | | | Brown | ++ | Good |
| Example 19 | | | | | |
| Example 24 | 15% | 2.4% | Brown | − | Good |
| Comparative | | | Brown | ++ | Good |
| Example 20 | | | | | |
| Example 25 | 20% | 3.2% | Brown | − | Good |
| Comparative | | | Brown | ++ | Good |
| Example 21 | | | | | |
| Example 26 | 30% | 4.8% | Brown | − | Good |
| Comparative | | | Brown | + | Good |
| Example 22 | | | | | |
| Example 27 | 50% | 8% | Dark brown | − | Good |
| Comparative | | | Dark brown | ± | Good |
| Example 23 | | | | | |
| Example 28 | 60% | 9.6% | Dark brown | − | Average |
| Comparative | | | Dark brown | ± | Average |
| Example 24 | | | | | |

Formation of Aggregate
++: Marked aggregation
+: Aggregation
±: Little aggregation
−: No aggregation
Capsule Strength
Good: No cracking or deformation in all 20 capsules
Average: Cracking or deformation in 1 to 2 capsules out of 20 capsules

As shown in Table 9, light to dark brown aggregates were observed in the capsule base of the hard capsules in Comparative Examples 17 to 24. This is in contrast to the analogous hard capsules of Examples 21 to 28, in which formation of aggregate was markedly suppressed. The hard capsules of the Examples were all brown in color, and had excellent transparency and glossiness.

### Experiment Example 4

200 g of hydroxypropyl methylcellulose (Shin-Etsu Chemical Co., Ltd.) was dispersed by stirring in 640 g of purified water kept at about 70° C. The dispersion liquid was cooled to 25° C. and hydroxypropyl methylcellulose was dissolved therein by stirring. Then, 160 g of an aqueous solution containing 40% methylcellulose and Caramel I (caramel SF-31, Ikedatohka Industries Co., Ltd.) that had been adjusted with a 0.1% citric acid aqueous solution (0.1% citric acid aqueous solutions containing varying concentrations—from 0.5% to 60%—of Caramel I) was added by stirring. The mixture was allowed to stand for seven hours for degassing. The resulting aqueous solution (capsule base aqueous solution) was used as an immersion liquid by charging it into a common capsule manufacturing apparatus using an immersion method, and the temperature of the immersion liquid was maintained at 39° C. to 41° C. The immersion liquid was then applied to a capsule mold heated to 80° C. and dried at 60° C. to prepare hard, brown capsules (Size 1; Examples 19 to 28).

As comparative examples, aqueous solutions containing 40% methyl cellulose and varying concentrations (0.5% to 60%) of Caramel I that had been adjusted with purified water instead of 0.1% citric acid aqueous solution were prepared. Using these aqueous solutions as pigments, hard, brown capsules were prepared as above (Comparative Examples 15 to 24).

The hard capsules were visually checked for a brown aggregate (caramel aggregate) as in Experiment Example 3. The results of the inspection are shown in Table 9, along with the results of the measurement of capsule strength.

TABLE 9

| | Concentration | Caramel | | | |
|---|---|---|---|---|---|
| | of Caramel I | concentration | Capsule | Formation of | Capsule |
| Examples | solution | in jelly | color | Aggregate | strength |
| Example 19 | 0.5% | 0.08% | Pale yellow | − | Good |
| Comparative | | | Pale yellow | − | Good |
| Example 15 | | | | | |
| Example 20 | 1% | 0.16% | Pale brown | − | Good |
| Comparative | | | Pale brown | − | Good |
| Example 16 | | | | | |
| Example 21 | 2% | 0.32% | Light brown | − | Good |
| Comparative | | | Light brown | + | Good |
| Example 11 | | | | | |
| Example 22 | 5% | 0.8% | Light brown | − | Good |
| Comparative | | | Light brown | ++ | Good |
| Example 18 | | | | | |
| Example 23 | 10% | 1.6% | Brown | − | Good |
| Comparative | | | Brown | ++ | Good |
| Example 19 | | | | | |
| Example 24 | 15% | 2.4% | Brown | − | Good |
| Comparative | | | Brown | ++ | Good |
| Example 20 | | | | | |
| Example 25 | 20% | 3.2% | Brown | − | Good |
| Comparative | | | Brown | ++ | Good |
| Example 21 | | | | | |
| Example 26 | 30% | 4.8% | Brown | − | Good |
| Comparative | | | Brown | + | Good |
| Example 22 | | | | | |
| Example 27 | 50% | 8% | Dark brown | − | Good |
| Comparative | | | Dark brown | ± | Good |
| Example 23 | | | | | |
| Example 28 | 60% | 9.6% | Dark brown | − | Average |
| Comparative | | | Dark brown | ± | Average |
| Example 24 | | | | | |

Formation of Aggregate
++: Marked aggregation
+: Aggregation
±: Little aggregation
−: No aggregation
Capsule Strength
Good: No cracking or deformation in all 20 capsules
Average: Cracking or deformation in 1 to 2 capsules out of 20 capsules

As shown in Table 9, light to dark brown aggregates were observed in the capsule base of the hard capsules in Comparative Examples 17 to 24. This is in contrast to the analogous hard capsules of Examples 21 to 28, in which formation of aggregate was markedly suppressed. The hard capsules of the Examples were all brown in color, and had excellent transparency and glossiness.

1. A brown film composition in the shape of a film or a sheet, which comprises a water-soluble cellulose derivative, caramel, and a pH adjuster.

2. A brown film composition according to claim 1, which is obtained by solidification of an aqueous solution containing a water-soluble cellulose derivative and caramel, wherein the aqueous solution forming a film contains a pH adjuster.

3. A brown film composition according to claim 1 or 2, wherein the caramel is at least one selected from the group consisting of Caramel I, Caramel II, and Caramel IV, and wherein the pH adjuster is an acid agent.

4. A brown film composition according to claim 2, wherein the caramel is at least one selected from the group consisting of Caramel I, Caramel II, and Caramel IV, and wherein the aqueous solution forming a film contains the pH adjuster so that the aqueous solution has a lower pH than an aqueous solution identical in composition except for the incorporation of the pH adjuster.

5. A brown film composition according to claim 1 or 2, wherein the caramel is Caramel III, and wherein the pH adjuster is an alkaline agent.

6. A brown film composition according to claim 2, wherein the caramel is Caramel III, and wherein the aqueous solution forming a film contains the pH adjuster so that the aqueous solution has a higher pH than an aqueous solution identical in composition except for the incorporation of the pH adjuster.

7. A container for food, medicine, cosmetics, agrichemicals, or feed, which comprises a brown film composition of any one of claims 1 through 6 at least partially.

8. A container according to claim 7, which is a form of a capsule.

9. A capsule formulation in which the capsule of claim 8 contains a filling.

10. A capsule formulation according to claim 9, wherein the filling is food, medicine, cosmetics, agrichemicals, or feed.

11. A method for preparing a brown film composition, said method comprising solidifying an aqueous solution containing a water-soluble cellulose derivative, caramel, and a pH adjuster.

12. A method according to claim 11, wherein the caramel is at least one selected from the group consisting of Caramel I, Caramel II, and Caramel IV, and wherein the pH adjuster is an acid agent.

13. A method according to claim 11, wherein the caramel is at least one selected from the group consisting of Caramel I, Caramel II, and Caramel IV, and wherein the aqueous solution containing a water-soluble cellulose derivative, caramel, and a pH adjuster is adjusted by using the pH adjuster in such a quantity that the aqueous solution has a lower pH than an aqueous solution identical in composition except for the incorporation of the pH adjuster.
